(19) United States
(12) Patent Application Publication
(10) Pub. No.: US 2018/0200177 A1
(43) Pub. Date: Jul. 19, 2018

(54) STRAIGHTENING AGENT CONTAINING A POLYMER COMBINATION OF POLYURETHANES AND AMODIMETHICONES

(71) Applicant: Henkel AG & Co. KGaA, Duesseldorf (DE)

(72) Inventors: Thorsten Knappe, Schenefeld (DE); Marie Meisel, Hemslingen (DE)

(73) Assignee: Henkel AG & Co. KGaA, Duesseldorf (DE)

(21) Appl. No.: 15/424,167

(22) Filed: Feb. 3, 2017

Related U.S. Application Data
(63) Continuation of application No. PCT/EP2015/064385, filed on Jun. 25, 2015.

(30) Foreign Application Priority Data
Aug. 6, 2014 (DE) 10 2014 215 486.3

(51) Int. Cl. A61K 8/87; A61K 8/898; A61K 8/81; A61Q 5/06

(52) U.S. Cl. CPC A61K 8/87; A61K 8/898; A61K 8/81; A61K 2800/594; A61Q 5/06; A61K 2800/5426; A61K 2800/5424; A61K 8/8164

(57) ABSTRACT

The invention relates to cosmetic agents for temporarily shaping keratin fibers, a method for temporarily shaping keratin fibers using said agents, and the use of said agents for temporarily shaping keratin fibers. The polymer combination of at least one specific anionic polyurethane polymer and a specific silicone-containing cationic copolymer used according to the invention results in excellent straightening properties and excellent protection of hair from damage during straightening.

### FIELD OF THE INVENTION

The present invention generally relates to the technical field of temporarily reshaping keratin-containing fibers, and in particular human hair.

The subject matter of the present invention is a cosmetic agent for temporarily reshaping keratin fibers, comprising in a cosmetically compatible carrier at least one specific anionic polyurethane polymer and at least one specific silicone-containing cationic copolymer.

A further subject matter of the present invention is a method for temporarily reshaping keratin fibers under the action of heat, wherein a cosmetic agent, comprising at least one specific anionic polyurethane polymer and at least one specific silicone-containing cationic copolymer, is used.

Finally, a further subject matter of the present invention is the use of a cosmetic agent, comprising at least one specific anionic polyurethane polymer and at least one specific silicone-containing cationic copolymer, for temporarily reshaping keratin fibers at a temperature of 30° C. to 250° C.

### BACKGROUND OF THE INVENTION

Keratin fibers, and in particular human hair, undergo a variety of treatments these days. Treatments that are used to impart a permanent or temporary shape to hair so as to obtain appealing hair styles play an important role in this process. Under prevailing fashion trends, fashionable hair styles often times are those that, in many hair types, can only be created or maintained for an extended period when certain setting active ingredients are used. Temporary shaping includes, for example, styling such as curling, straightening, teasing or also setting.

Temporary shaping that is to yield strong hold without impairing the healthy appearance of the hair, such the shine thereof, can be achieved by styling agents, such as hair sprays, hair waxes, hair gels, hair setting lotions, blow drying lotions, styling sprays and the like.

In the field of temporary shaping of hair, hair reshaping under the action of heat, for example by way of a blow dryer, a curling iron or a straightening iron, likewise plays an important role. So as to improve styling results, a styling agent, such as a hair setting lotion or a styling spray, is often applied to the hair prior to the application of heat, and in the case of higher temperatures, also what is known as a thermal styling spray is applied. Especially so-called straightening irons (hot irons) are used increasingly in the hairdresser field. Hair straighteners comprise two parallel metal or ceramic plates, through which the hair is pulled after the plates have been heated by guiding the straightening iron along a strand of hair. Commercially available hair straighteners can be heated to temperatures in the range of 150° C. to 250° C. The goal of using a hair straightener is to thermally/physically straighten wavy to curly hair.

If hair is to be straightened by way of a hair straightener or a blow dryer, usually a thermal styling agent, also referred to as a straightener agent, is applied to the hair beforehand. The agent is intended to support the sliding motion of the iron and the straightening of the hair. Furthermore, these agents are intended to prevent or minimize damage to the hair as a result of the high temperatures used during straightening.

The styling agents known from the prior art, and in particular thermal styling agents, however, do not always allow satisfactory straightening to be achieved. Moreover, the styling agents known from the prior art still have room for improvement when it comes to minimizing damage to the hair.

It was the object of the present invention to provide cosmetic agents for temporarily reshaping keratin fibers which have an improved styling result in the form of a high straightening action and reduced damage to the hair or improved care, and in particular improved wet and dry combability. These cosmetic agents should furthermore have a long shelf life.

Surprisingly, it was found that cosmetic agents for temporarily reshaping keratin fibers ensure a high straightening action and reduced damage to the hair if these agents comprise a polymer combination of a specific anionic polyurethane polymer and a specific silicone-containing cationic copolymer. In addition, no incompatibilities occur when these specific polymers are combined, ensuring a long shelf life of the cosmetic agents according to the invention.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic agent for temporarily reshaping keratin fibers, comprising in a cosmetically compatible carrier a) at least one anionic polyurethane polymer, comprising at least one structural unit of formula (I), at least one structural unit of formula (II), at least one structural unit of formula (III), at least one structural unit of formula (IV), and at least one structural unit of formula (V)

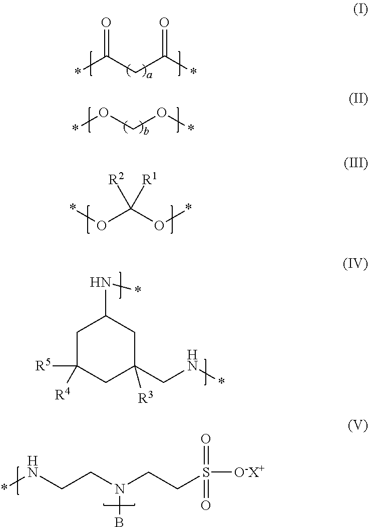

where a and b, independently of one another, denote integers from 1 to 20; R¹, R², R³, R⁴ and R⁵, independently of one another, denote a hydrogen atom or a C₁ to C₄ alkyl group; B denotes a hydrogen atom or a direct bond with a further structural unit; X⁺ denotes a physiologically compatible cation; and b) at least one silicone-containing cationic copolymer, comprising at least one structural unit of formula (IV), at least one structural unit of formula (VII), and at least one structural unit of formula (VIII)

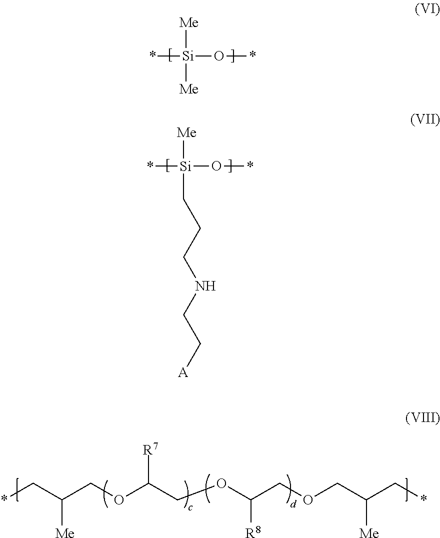

where A denotes a hydroxyl group, an amino group or a thiol group; R⁷ and R⁸, independently of one another, denotes a hydrogen atom, a methyl group, an ethyl group, a C₁-C₄ aralkyl group, C₂-C₆ alkenyl group, or a C₂-C₆ hydroxyalkyl group; and c and d, independently of one another, denote integers from 10 to 55.

A cosmetic agent for temporarily reshaping keratin fibers, comprising in a cosmetically compatible carrier a) at least one anionic polyurethane polymer of adipic acid, 1,6-hexanediol, nepentylglycol, isophorone dicyanate, isophorone diamine and the sodium salt of N-(2-aminoethyl)-3-aminoethane sulfonic acid; and b) at least one silicone-containing cationic copolymer of polyethylene-polypropylene glycol-bis(2-methyl-2-propene-1-yl)ether, 3-[(2-aminoethyl)amino]propyl-siloxane and dimethylsiloxane.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention relates to a cosmetic agent for temporarily reshaping keratin fibers, comprising, in a cosmetically compatible carrier,
a) at least one anionic polyurethane polymer, comprising at least one structural unit of formula (I), at least one structural unit of formula (II), at least one structural unit of formula (III), at least one structural unit of formula (IV), and at least one structural unit of formula (V)
b)

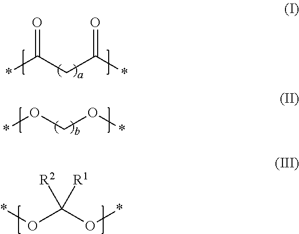

c)

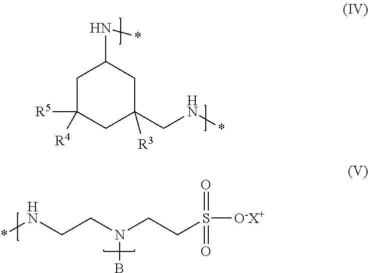

where
a and b, independently of one another, denote integers from 1 to 20;
R¹, R², R³, R⁴ and R⁵, independently of one another, denote a hydrogen atom or a C₁ to C₄ alkyl group;
B denotes a hydrogen atom or a direct bond with a further structural unit;
X⁺ denotes a physiologically compatible cation; and
a) at least one silicone-containing cationic copolymer, comprising at least one structural unit of formula (VI), at least one structural unit of formula (VII), and at least one structural unit of formula (VIII)

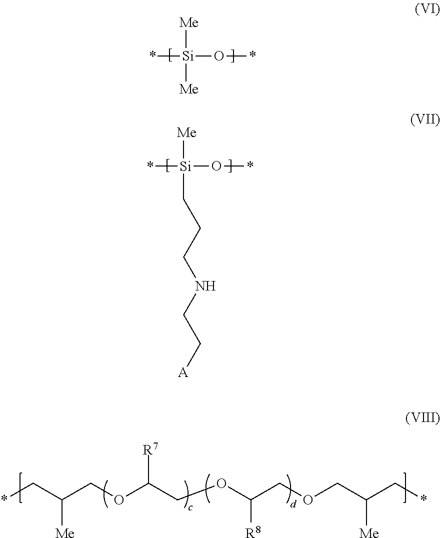

where
A denotes a hydroxyl group, an amino group or a thiol group;
R⁷ and R⁸, independently of one another, denote a hydrogen atom, a methyl group, an ethyl group, a C₁-C₄ aralkyl group, C₂-C₆ alkenyl group, or a C₂-C₆ hydroxyalkyl group; and
c and d, independently of one another, denote integers from 10 to 55.

The anionic polyurethane polymer used in the cosmetic agents according to the invention has excellent film-forming properties and thus ensures a firm hold of the hair style and a long-lasting straightening result. However, the sole use of this anionic polyurethane polymer results in only inadequate protection against hair damage under the high temperatures that occur during straightening, and thus does not result in satisfactory care of the straightened hair. To increase the care during straightening, the use of further cationic compounds, in particular those known in the prior art for the nourishing action thereof, is thus necessary. However, it has been shown that, due to incompatibilities, the anionic polyurethane polymer cannot be used in combination with the cationic nourishing substances typically used to improve hair care. It was therefore surprising that the anionic polyurethane polymer can be combined with nourishing substances in the form of specific silicone-containing cationic copolymers, without causing incompatibilities between these polymers that result in a reduced shelf life of the cosmetic agents according to the invention or adversely affect the high straightening action of the anionic polyurethane polymer and the high heat protection action or nourishing action of the silicone-containing cationic copolymer.

According to the above formulas and all formulas provided hereafter, a chemical bond identified by the “*” symbol denotes a free valence of the corresponding structure fragment. Free valence here shall be understood to mean the number of atom bonds originating from the corresponding structure fragment at the position identified with the “*” symbol. Within the scope of the present invention, preferably a respective atomic bond extends from the positions of the structure fragments identified by the “*” symbol to further structural fragments.

In principle, keratin fibers shall be understood to mean all animal hair, such as wool, horsehair, angora wool, furs, feathers, and products or textiles produced therefrom. The keratin fibers, however, are preferably human hair.

Furthermore, the term “polyurethane polymers” shall be understood to mean polymers in which at least two monomers, and preferably at least three monomers, are linked by a urethane grouping —NH—CO—O—. According to the invention also covered are polyurethane polymers that, by virtue of the production process, comprise urea group-containing repeating units —NH—C(O)—NH—, as they are formed in particular during the reaction of isocyanate-terminated prepolymers with amino group-containing compounds. Particularly preferred according to the invention are polyurethane polymers in which all monomers are linked via urethane groupings and/or urea group-containing groupings. Such polyurethane polymers can be obtained, for example, by reacting dihydric or polyhydric alcohols with diisocyanates.

Moreover, according to the invention the term “anionic polyurethane polymers” shall be understood to mean those polyurethane polymers that in a protic solvent, under standard conditions, carry at least one structural unit comprising permanently anionic groups, wherein the anionic groups must be compensated for by counterions, preserving the electroneutrality. According to the invention, in particular carboxyl and sulfonic acid groups fall under anionic groups.

Within the scope of the present invention, additionally the term “silicone-containing cationic copolymers” shall be understood to mean copolymers that comprise at least one grouping —Si(R)₂—O— and in a protic solvent, under standard conditions, carry at least one structural unit comprising a cationic group, wherein the cationic groups likewise must be compensated for by counterions, preserving the electroneutrality. The silicone-containing cationic copolymers are preferably silicone-containing copolymers that comprise cationic groups in the form of protonated amines.

Furthermore, the term “fatty acid”, as it is used within the scope of the present invention, shall be understood to mean aliphatic carboxylic acids that include unbranched or branched carbon groups having 4 to 40 carbon atoms. The fatty acids used within the scope of the present invention can be both naturally occurring and synthetically produced fatty acids. The fatty acids can moreover be monounsaturated or polyunsaturated.

Finally, the term “fatty alcohol” within the scope of the present invention shall be understood to mean aliphatic, monohydric, primary alcohols that include unbranched or branched hydrocarbon groups having 4 to 40 carbon atoms. The fatty alcohols used within the scope of the invention can also be monounsaturated or polyunsaturated.

The agents according to the invention comprise a cosmetic carrier. According to the invention, the cosmetic carrier is preferably aqueous, alcoholic or aqueous-alcoholic.

An aqueous carrier within the meaning of the invention comprises at least 30 wt. %, and in particular at least 50 wt. %, water, based on the total weight of the cosmetic agent.

Aqueous-alcoholic carriers within the meaning of the present invention shall be understood to mean compositions including water and a total amount of 3 to 90 wt. % of a C₁-C₄ alcohol, based on the total weight of the cosmetic agent, in particular ethanol or isopropanol.

It may be preferred according to the invention if at least one (C₂-C₆) alkyl alcohol comprising at least one hydroxyl group is used as an additional solvent of the agent according to the invention. Within the scope of a particularly preferred embodiment of the agent according to the invention, the agent includes at least one alcohol comprising 2 to 6 carbon atoms and 1 to 3 hydroxyl groups as an additional solvent.

The solvent, which is different from water, is preferably selected from at least one compound of the group consisting of ethanol, ethylene glycol, isopropanol, 1,2-propylene glycol, 1,3-propylene glycol, glycerol, n-butanol, 1,3-butylene glycol. Ethanol is an especially particularly preferred solvent.

Polyethylene glycol and/or polypropylene glycol are further particularly preferred solvents.

In particular, the addition of polyethylene glycol and/or polypropylene glycol increases the flexibility of the polymer film that is formed when using the cosmetic agent according to the invention. If flexible hold is desired, the cosmetic agents according to the invention preferably have a total content of 0.01 to 30 wt. % of polyethylene glycol and/or polypropylene glycol, based on the total weight of the cosmetic agent.

As a first essential component a), the cosmetic agent according to the invention comprises at least one specific anionic polyurethane polymer based on structural units of formulas (I) to (V). In the structural units of formulas (III) and (IV), the groups R¹ to R⁵ can denote (C₁-C₄) alkyl groups. Examples of these are methyl, ethyl, propyl, isopropyl, butyl, sec-butyl, isobutyl and tert-butyl groups.

The anionic polyurethane polymers present in the cosmetic agents according to the invention can be obtained by reacting an isocyanate group-containing prepolymer (V1) with at least one amino group-containing compound (V2).

For this purpose, preferably isocyanate group-containing prepolymers (V1) that comprise terminal isocyanate groups are used, which is to say these polymers (V1) comprise isocyanate groups at the chain ends, and preferably at all chain ends, of the polymer. It is furthermore preferred if isocyanate group-containing prepolymers are used that are obtained by reacting isocyanates or polyisocyanates with one or more polyols, selected from the group consisting of polyether polyols, polycarbonate polyols, polyether-polycarbonate polyols and/or polyester polyols. Suitable prepolymers (V1) are thus in particular the aliphatic, aromatic or cycloaliphatic polyisocyanates having an NCO functionality of greater than or equal to 2, which are known per se to a person skilled in the art.

Particularly preferred isocyanates are hexamethylene diisocyanate, isophorone diisocyanate or the isomeric bis-(4,4′-isocyanatocyclohexyl)methanes, and mixtures of the aforementioned diisocyanates. Particularly preferred aliphatic polyester polyols are polyester polyols based on aliphatic carboxylic acids and aliphatic polyols, in particular based on adipic acid and aliphatic alcohols, such as hexanediol and/or neopentyl glycol having an average molecular weight Mw of 600 to 3000 g/mol. Furthermore, preferably polycarbonates, and in particular polydicarbonate diols, comprising at least one hydroxyl group and having an average molecular weight Mw of 600 to 3000 g/mol are used, which are obtainable, for example, by reacting carbonic acid derivatives, such as diphenyl carbonate, dimethyl carbonate or phosgene, with polyols, and preferably diols. Polyether polyols, such as polytetramethylene glycol polyethers and polycarbonate polyols, or the mixtures thereof, can likewise preferably be used. The average molecular weight Mw can be determined, for example, by way of gel permeation chromatography (GPC) using polystyrene as the internal standard, as described in the German unexamined patent application DE 1999614603.

The amino group-containing compounds (V2) used to produce the anionic polyurethane polymers used according to the invention are preferably selected from primary and/or secondary amines and/or diamines. In particular, the amino group-containing compounds (V2) comprise at least one diamine. The amino group-containing compounds (V2) are preferably selected from amino group-containing compounds that comprise ionic or ionogenic (ion-forming) groups and from amino group-containing compounds that do not comprise any ionic or ionogenic groups. Suitable amino group-containing compounds (V2) that do not comprise any ionic or ionogenic groups are preferably selected from 1,2-ethylenediamine, bis(4-aminocyclohexyl)methane, 1,4-diaminobutane, isophoronediamine, ethanolamine, diethanolamine and diethylenetriamine. Amino group-containing compounds (V2) that comprise ionic and/or ionogenic groups preferably comprise sulfonate or sulfonic acid groups, and particularly preferably sodium sulfonate groups. Amino group-containing compounds (V2) that comprise ionic or ionogenic groups are preferably selected from salts of 2-(2-aminoethylamino)ethanesulfonic acid, ethylenediamine propyl or -butyl sulfonic acid, 1,2- or 1,3-propylenediamine-β-ethylsulfonic acid and taurine.

According to a first embodiment of the present invention, in the structural unit of formula (I) a denotes integers from 1 to 10, preferably from 2 to 8, and in particular from 2 to 6, and in the structural unit of formula (II) b denotes integers from 1 to 12, preferably from 2 to 10, and in particular from 4 to 8.

Within the scope of the present invention, it may furthermore be provided that in the structural units of formulas (III) and (IV) the groups R¹, R², R³, R⁴ and R⁵, independently of one another, denote hydrogen or a methyl group, and in particular a methyl group.

It is preferred according to the invention if in the structural unit of formula (I) X⁺ denotes metal cations of the physiologically compatible metals from groups IA, IB, IIA, IIB, IIIB, VIA or VIII of the periodic system of elements, ammonium ions, and cationic organic compounds comprising a quaternized nitrogen atom. For example, cationic organic compounds can be obtained by protonating primary, secondary or tertiary organic amines with an acid or by permanently quaternizing these organic amines. Examples of cationic organic ammonium compounds suitable within the scope of the present invention are 2-ammoniethanol and 2-trimethylammonioethanol, for example.

According to a particularly preferred embodiment of the present invention, the cosmetic agent according to the invention comprises at least one anionic polyurethane polymer a), which comprises at least one structural unit of formula (Ia), at least one structural unit of formula (IIa), at least one structural unit of formula (IIIa), at least one structural unit of formula (IVa) and at least one structural unit of formula (Va),

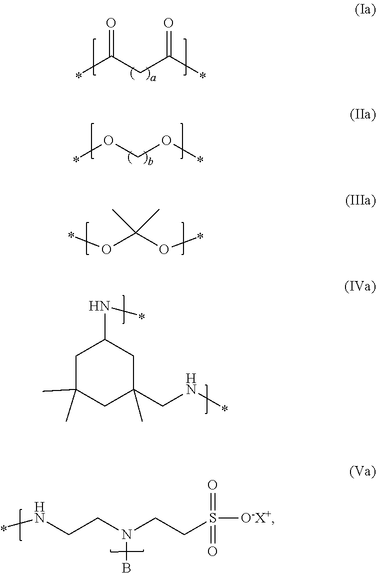

where
a denotes integers from 2 to 6;
b denotes integers from 4 to 8; and
X⁺ denotes a physiologically compatible cation, in particular sodium. The use of the above-mentioned specific anionic polyurethane polymers in the cosmetic agents according to the invention results in outstanding hold of the hair style, and in particular in an outstanding straightening action. In addition, the outstanding hold of the hair style is ensured over a long period of time.

An especially particularly preferred anionic polyurethane polymer within the scope of the present embodiment is the polymer known under the INCI name Polyurethane-48. This polymer includes adipic acid (a in the structural unit of formula (Ia) denotes the integer 4) and 1,6-hexanediol (b according to the structural unit of formula (IIa) denotes the integer 6).

Anionic polyurethane polymers that are preferably used according to the invention have a certain glass transition temperature T_{g}. It is thus preferred within the scope of the present invention if the at least one anionic polyurethane polymer a) has a glass transition temperature T_{g} of −70° C. to −20°, especially of −65° C. to −25° C., preferably of −60° C. to −30° C., and in particular of −50° C. to −40° C. The glass transition temperatures are determined by way of differential scanning calorimetry (DSC) at a heating rate of 10 K/min, a starting temperature of at least 30° C. below the glass transition temperature, and an ending temperature of at least 30° C. above the glass transition temperature. The glass transition temperature T_{g} is derived as the midpoint temperature according to the tangent method.

Preferred cosmetic agents according to the invention include the at least one anionic polyurethane polymer a) in a total amount of 0.05 to 5 wt. %, especially 0.08 to 3 wt. %, preferably 0.1 to 2 wt. %, and in particular 0.2 to 1.5 wt. %, based on the total weight of the cosmetic agent. The use of the above-mentioned amounts of the specific anionic polyurethane polymer a) results in an outstanding straightening action of the cosmetic agent according to the invention. In addition, using these amounts ensures that no negative interactions with other ingredients occur, in particular with the silicone-containing cationic copolymer, which can adversely affect the shelf life and the straightening action of the cosmetic agents according to the invention.

As a second essential component b), the cosmetic agent according to the invention comprises at least one specific silicone-containing cationic copolymer based on structural units of formulas (VI) to (VIII). The use of this silicone-containing cationic copolymer results in a high nourishing action and in high protection against damage to the keratin fibers shaped, and in particular straightened, by way of the cosmetic agents according to the invention.

With respect to the nourishing action, and in particular the decrease in damage to the hair, of the cosmetic agents according to the invention, it has proven advantageous if the group A in the structural unit of formula (VII) denotes an amino group.

Particularly good results are obtained within the scope of the present invention if the groups R⁷ and R⁸ in the structural unit of formula (VIII), independently of one another, denote a hydrogen atom or a methyl group, and preferably R⁷ denotes hydrogen and R⁸ denotes a methyl group.

It is furthermore particularly preferred within the scope of the present invention if in the structural unit of formula (VIII) c denotes integers from 15 to 30, and in particular from 15 to 25, and d denotes integers from 20 to 45, and preferably from 25 to 40.

Within the scope of the present invention, it is thus especially particularly preferred if the cosmetic agent comprises at least one cationic silicone-containing copolymer that comprises at least one structural unit of formula (VIa), at least one structural unit of formula (VIIa) and at least one structural unit of formula (VIIIa)

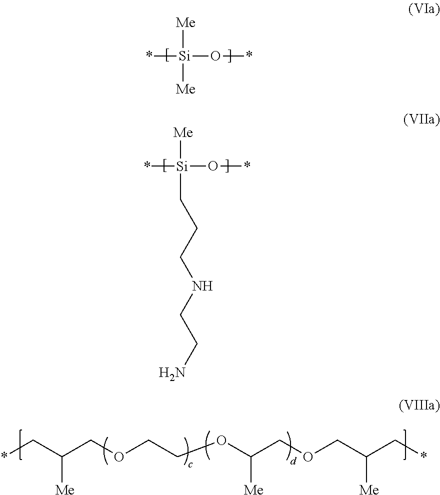

where
c denotes integers from 15 to 25; and
d denotes integers from 25 to 40. Using the above-mentioned specific silicone-containing cationic copolymers in the cosmetic agents according to the invention results in outstanding protection of the hair against damage that may occur due to the high temperatures used during straightening, and in outstanding care, in particular in improved wet and/or dry combability. In addition, these specific silicone-containing cationic copolymers exhibit no negative interactions with the specific anionic polyurethane polymers, whereby a long shelf life, improved protection against damage to the hair, and improved care of the cosmetic agents according to the invention are ensured.

An especially particularly preferred cationic silicone-containing copolymer within the scope of the present embodiment is a polymer bearing the INCI name Bis-Isobutyl PEG/PPG-20/35/Amodimethicone Copolymer. This polymer includes approximately 20 moles ethylene oxide (c in the structural unit of formula (VIIa) denotes the integer 20) and approximately 35 moles propylene oxide (d according to the structural unit of formula (VIIIa) denotes the integer 35).

Preferred cosmetic agents according to the invention comprise the at least one silicone-containing copolymer b) in a total amount of 0.01 to 2 wt. %, especially 0.02 to 1.5 wt. %, preferably 0.03 to 1 wt. %, and in particular 0.05 to 0.5 wt. %, based on the total weight of the cosmetic agent. Using the above-mentioned amounts of the specific silicone-containing cationic copolymer b) results in an outstanding nourishing action, and in particular in improved wet and/or dry combability, and in outstanding protection against damage to the hair of the cosmetic agents according to the invention. In addition, using these amounts ensures that no negative interactions with other ingredients occur, in particular with the anionic polyurethane polymer, which can adversely affect the shelf life, the straightening action, and the protection against damage to the hair of the cosmetic agents according to the invention.

Cosmetic agents according to the present invention, comprising in a cosmetically compatible carrier
a) at least one anionic polyurethane polymer, comprising at least one structural unit of formula (Ia), at least one structural unit of formula (IIa), at least one structural unit of formula (IIIa), at least one structural unit of formula (IVa), and at least one structural unit of formula (Va)

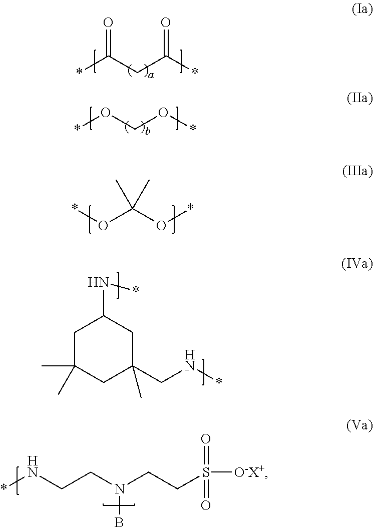

where
a denotes integers from 2 to 6;
b denotes integers from 4 to 8; and
X⁺ denotes metal cations of the physiologically compatible metals from groups IA, IB, IIA, IIB, IIIB, VIA or VIII of the periodic system of elements, ammonium ions, and cationic organic compounds comprising a quaternized nitrogen atom; and
b) at least one cationic silicone-containing copolymer that comprises at least one structural unit of formula (VIa), at least one structural unit of formula (VIIa) and at least one structural unit of formula (VIIIa)

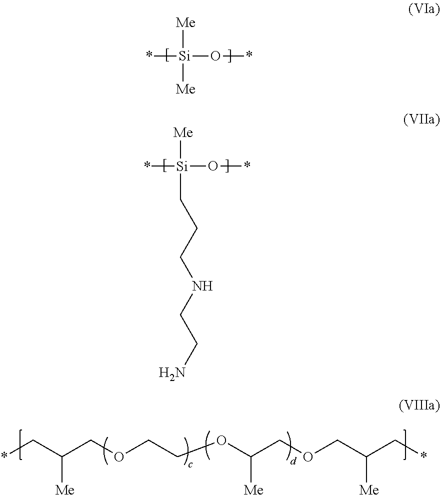

where
c denotes integers from 15 to 25; and
d denotes integers from 25 to 40
   are particularly preferred within the scope of the present invention. Using the above-mentioned specific polymer combination is particularly advantageous with respect to outstanding hold of the hair style, in particular in the form of an outstanding straightening action, and outstanding protection of the hair against damage and outstanding care, in particular wet and/or dry combability. Moreover, the combination of the above-mentioned specific polymers does not lead to any negative interactions that result in a reduced shelf life and in a reduced straightening action and nourishing action of the cosmetic agents according to the invention.

Particularly good results within the scope of the present invention are obtained when the cosmetic agent has a weight ratio of the total amount of the at least one anionic polyurethane polymer a) to the total amount of the at least one silicone-containing copolymer b) of 300:1 to 1:40, especially of 100:1 to 1:20, preferably of 40:1 to 1:5, more preferably of 20:1 to 1:1, and in particular of 10:1 to 2:1. At the above-mentioned weight ratios, no negative interactions occur between the specific anionic polyurethane polymer and the specific silicone-containing cationic copolymer, so that the cosmetic agents according to the invention exhibit both the outstanding straightening properties of the anionic polyurethane polymer and the outstanding nourishing properties and the outstanding protective properties of the silicone-containing cationic copolymers. Furthermore, due to the absence of unfavorable negative interactions between the specific polymers, cosmetic agents according to the invention have a long shelf life at the above-mentioned weight ratios.

Within the scope of the present invention, it is preferred for the cosmetic agent to have a pH value of pH 3.0 to pH 7.0, especially of pH 3.5 to pH 6.0, and in particular of pH 4.0 to pH 5.5. These pH values ensure that the silicone-containing copolymer is present in the form of a cationic compound and has a high affinity to the keratin fibers. The high affinity to the keratin fibers ensures even coverage of these fibers and thus effective protection of these fibers against damage by the high temperatures used during straightening. The pH value can be set using acids and bases customarily used in styling agents, preferably organic acids, such as lactic acid and/or citric acid.

The straightening action and the protection of keratin fibers against damage to the hair can be further enhanced if the cosmetic agent additionally comprises an anionic polymer, comprising at least one structural unit of formula (IX) and at least one structural unit of formula (X)

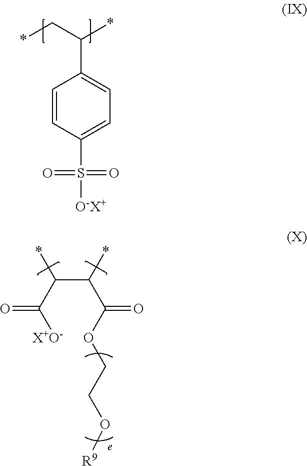

where
R⁹ denotes a linear or branched, saturated or unsaturated C₁₀-C₄₀ alkyl group or a C₁₀-C₄₀ hydroxyalkyl group, and in particular a linear or branched, saturated or unsaturated C₁₄-C₃₆ alkyl group;
e denotes integers between 20 and 70, especially 30 and 60, and in particular 35 and 45; and
X⁺ denotes a physiologically compatible cation.

The long-lasting hold of the cosmetic agent according to the invention can be further enhanced if the cosmetic agent additionally comprises at least one film-forming and/or setting polymer, which is different from the anionic polyurethane polymer a). Film-forming or setting polymers contribute to the hold of the shape imparted to the collective fibers, such as the overall hair style, by the formation of a film. The film formation can also take place in a punctiform manner and join only a few of the fibers to one another. Film-forming or setting polymers shall be understood to mean polymers that, on drying, leave behind a continuous film on the skin, the hair or the nails. Such film formers can be used in a wide variety of cosmetic products, such as facial masks, make-up, hair setting lotions, hair sprays, hair gels, hair waxes, deep hair conditioners, shampoos or nail polishes. In particular, polymers that exhibit sufficient solubility in water or water/alcohol mixtures are preferred, so as to be present in fully dissolved form in the cosmetic agents according to the invention. The film-forming polymers may be of synthetic or natural origin. Film-forming polymers are furthermore also understood to include polymers that, when used in a 0.01 to 20 wt. % aqueous, alcoholic or aqueous-alcoholic solution, are capable of depositing a transparent polymer film on the hair.

The additional setting polymers are preferably selected from non-ionic setting polymers, amphoteric setting polymers, cationic setting polymers and anionic setting polymers, and particularly preferably from non-ionic setting polymers, anionic setting polymers, and amphoteric setting polymers.

The agent according to the invention preferably additionally comprises at least one setting non-ionic polymer, comprising at least one structural unit from the group of structural units of formulas (P1) to (P6)

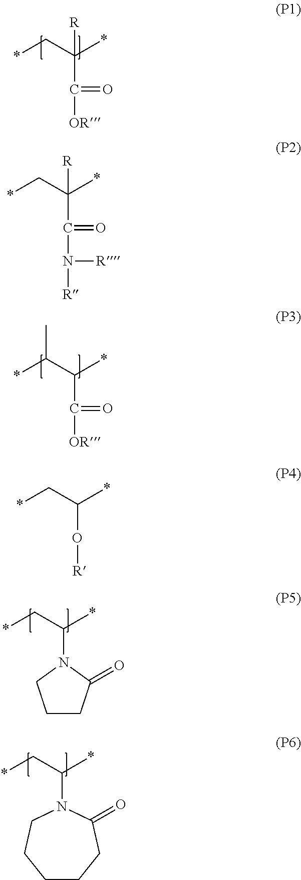

where
R denotes a hydrogen atom or a methyl group;
R′ denotes a hydrogen atom or a (C₁ to C₄) acyl group;
R″ and R″″, independently of one another, denote a (C₁ to C₇) alkyl group or a hydrogen atom; and
R′″ denotes a linear or branched (C₁ to C₄) alkyl group or a (C₂ to C₄) hydroxyalkyl group.

Preferred non-ionic setting polymers that comprise at least one of the above-mentioned structural units (P1) to (P6) are homopolymers or copolymers composed of at least one of the following monomers: N-vinylpyrrolidone, N-vinylcaprolactam, vinyl esters (such as vinyl acetate, vinyl alcohol), acrylamide, methacrylamide, alkyl and dialkyl acrylamide (in particular N-methyl and N,N-dimethyl acrylamide), alkyl and dialkyl methacrylamide (in particular N-methyl and N,N-dimethyl methacrylamide), alkyl acrylate, alkyl methacrylate, wherein the alkyl groups of these monomers are each selected from (C₁ to C₃) alkyl groups.

Particularly suited non-ionic polymers based on ethylenically unsaturated monomers comprise at least one of the following structural units

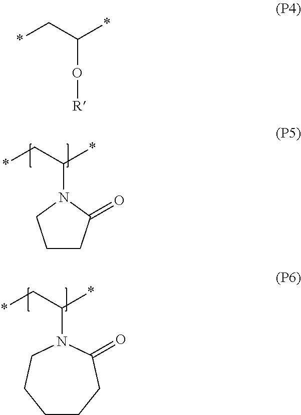

where R′ denotes a hydrogen atom or a (C₁ to C₃₀) acyl group, and in particular a hydrogen atom or an acetyl group.

Suitable polymers, comprising at least one of the structural units (P4) to (P6), are in particular homopolymers of vinylcaprolactam or of vinylpyrrolidone (such as Luviskol® K 90 or Luviskol® K 85 from BASF SE), copolymer products of vinylpyrrolidone and vinyl acetate having a molar ratio of the structural units obtained from the monomer N-vinylpyrrolidone to the structural units obtained from the monomer vinyl acetate in the range of 20:80 to 80:20, and in particular of 30:70 to 60:40 (for example, sold under the trademark Luviskol® VA 37, Luviskol® VA 55, Luviskol® VA 64 and Luviskol® VA 73 by BASF SE), terpolymers of vinylpyrrolidone, vinyl acetate and vinyl propionate, polyacrylamides (such as Akypomine® P 191 from CHEM-Y), polyvinyl alcohols (which are sold, for example, by Du Pont under the trade name Elvanol® or by Air Products under the trade name Vinol® 523/540), terpolymers of vinylpyrrolidone, methacrylamide and vinylimidazole (such as Luviset® Clear from BASF SE).

Cosmetic agents according to the invention that comprise, as the setting non-ionic polymer, at least one polymer selected from the group consisting of
polyvinylpyrrolidone,
copolymers of N-vinylpyrrolidone and vinyl esters of carboxylic acids having 2 to 18 carbon atoms, in particular of N-vinylpyrrolidone and vinyl acetate,
   are especially particularly preferred.

Within the scope of a further embodiment, the cosmetic agent according to the invention comprises at least one setting anionic polymer as an additional setting polymer.

Preferred setting anionic polymers comprise at least one structural unit of formula (P7) and at least one structural unit of formula (P8),

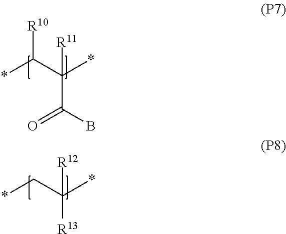

where
R¹⁰ and R¹¹, independently of one another, denote a hydrogen atom or a methyl group, with the proviso that R¹⁰ and R¹¹ do not simultaneously denote a methyl group;
R¹² denotes a hydrogen atom or a methyl group;
R¹³ denotes a carbamoyl group, a linear or branched (C₄ to C₁₂) alkylaminocarbonyl group, a linear or branched (C₄ to C₁₂) alkyloxycarbonyl group, a linear or branched (C₂ to C₁₂) acyloxy group, a (C₂ to C₄) hydroxyalkylcarbonyl group, or a phenyl group, and
B denotes a hydroxy group or an organic group comprising at least one sulfonic acid group, which binds to the structural unit via an oxygen atom or an NH group.

In this connection, it may be preferred for the anionic setting polymer to comprise at least one structural unit of formula (P7), which is selected from at least one structural unit of formulas (P7-I) to (P7-V).

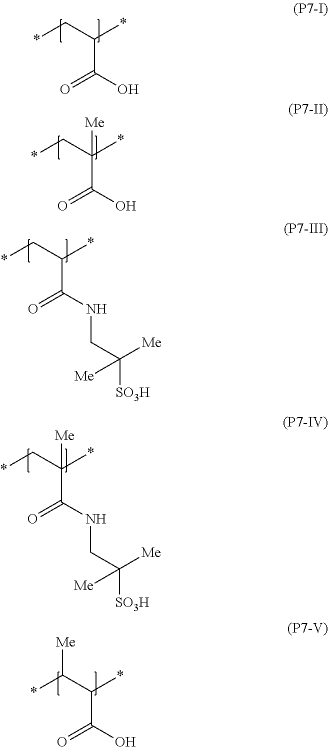

It is particularly preferred according to the invention for the anionic setting polymer to comprise at least one structural unit of formula (P8), which is selected from at least one structural unit of formulas (P8-I) to (P8-VII)

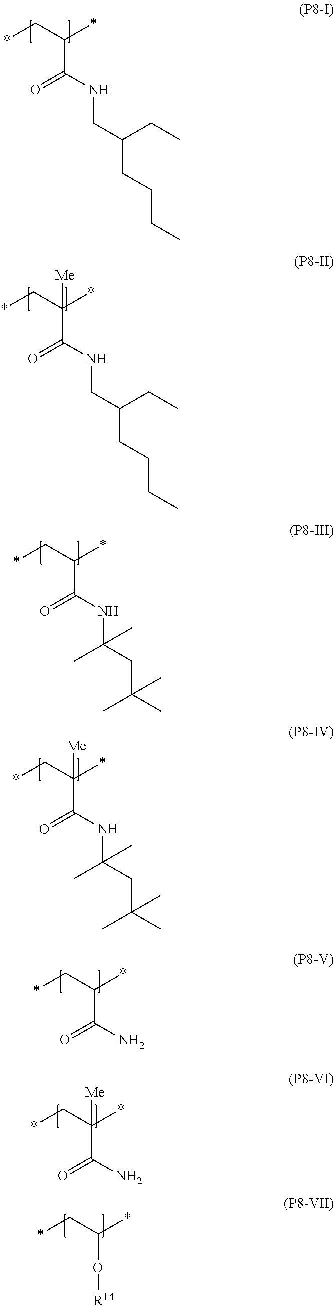

where
R¹⁴ denotes a (C₂ to C₁₂) acyl group, and in particular acetyl or neodecanoyl.

Moreover, it is preferred for the anionic setting polymer to comprise, in addition to the above structural units of formulas (P7) and (P8), also at least one structural unit of formula (P9)

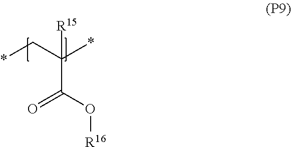

where
R¹⁵ denotes a hydrogen atom or a methyl group; and
R¹⁶ denotes a (C₁ to C₃) alkyl group, and in particular a methyl group or an ethyl group.

In general, it is particularly preferred if group B is present in all the above structural units of formula (P7) in partially or completely neutralized form. Such cosmetic agents according to the invention thus additionally comprise at least one alkanolamine. The alkanolamines that can be used as alkalizing agents according to the invention are preferably selected from primary amines having a C₂-C₆ alkyl base body, which carries at least one hydroxyl group. Particularly preferred alkanolamines are selected from the group consisting of 2-aminoethane-1-ol (monoethanolamine), 3-aminopropane-1-ol, 4-aminobutane-1-ol, 5-aminopentane-1-ol, 1-aminopropane-2-ol, 1-aminobutane-2-ol, 1-aminopentane-2-ol, 1-aminopentane-3-ol, 1-aminopentane-4-ol, 3-amino-2-methylpropane-1-ol, 1-amino-2-methylpropane-2-ol, 3-aminopropane-1,2-diol, 2-amino-2-methylpropane-1,3-diol. Especially particularly preferred alkanolamines according to the invention are selected from the group consisting of 2-aminoethane-1-ol, 2-amino-2-methylpropane-1-ol and 2-amino-2-methyl-propane-1,3-diol.

Cosmetic agents according to the invention that comprise, as the setting anionic polymer, at least one polymer selected from the group consisting of
copolymers of methacrylic acid and ethyl acrylate and tert-butyl acrylate (available under the trade names Luvimer 100 P, Luvimer 30 E or Luvimer 36 D from BASF SE),
copolymers of acrylic acid and ethyl acrylate and tert-butyl acrylamide (available under the trade names Ultrahold Strong or Ultrahold 8 from BASF SE),
copolymers of 2-acrylamido-2-methyl-propanesulfonic acid and acrylamide,
copolymers of 2-acrylamido-2-methyl-propanesulfonic acid, acrylamide and acrylic acid,
copolymers of acrylamide with methacrylic acid and acryloyldimethyltaurate (available under the trade name Acudyne® SCP from Rohm & Haas),
copolymers of vinyl acetate and crotonic acid (available under the trade name Aristoflex A 60 from Clariant),
copolymers of vinyl propionate and crotonic acid (available under the trade name Luviset CA 66 from BASF SE),
copolymers of vinyl neodecanoate, vinyl acetate and crotonic acid (available under the trade names Resyn 28-2942 and Resyn 28-2930 from National Starch),
   are especially particularly preferred.

Furthermore, it may be provided according to the invention that the agent according to the invention comprises at least one amphoteric setting polymer as the setting polymer.

Preferred amphoteric setting polymers comprise at least one structural unit of formula (P10) and at least one structural unit of formula (P11),

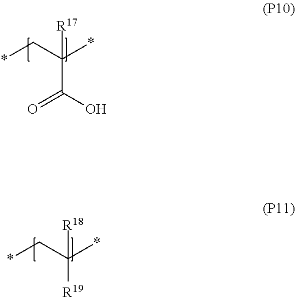

where
R¹⁸ and R¹⁹, independently of one another, denote a hydrogen atom or a methyl group;
R¹⁷ denotes a linear or branched (C₄ to C₁₂) alkylaminoethyl-aminocarbonyl group, a linear or branched (C₄ to C₁₂) alkylaminopropyl-aminocarbonyl group, a linear or branched (C₄ to C₁₂) alkylaminoethyloxycarbonyl group, a linear or branched (C₄ to C₁₂) alkylaminopropyloxycarbonyl group.

It is particularly preferred if the structural unit of formula (P10) is present in entirely or partially neutralized form. Such cosmetic agents according to the invention thus additionally comprise at least one alkanolamine. Suitable alkalizing agents are the alkalizing agents listed above in connection with anionic setting polymers. The amount of alkalizing agent used in the cosmetic agents according to the invention is preferably 80 to 100%, preferably 90 to 100%, and in particular 95 to 100%/o of the amount required for full neutralization of the additional amphoteric setting polymers.

Particularly suited is at least one amphoteric setting polymer that, in addition to at least one of the above structural units of formulas (P10) and (P11), also comprises at least one structural unit of formula (P12)

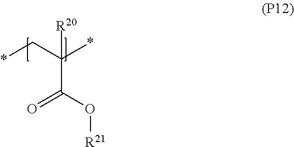

where
R²⁰ denotes a hydrogen atom or a methyl group; and
R²¹ denotes a (C₁ to C₄) alkyl group, and in particular a methyl group or an ethyl group.

Preferred amphoteric setting polymers of this type are selected from the group formed of copolymers of acrylic acid, at least one (C₁ to C₄) alkyl acrylate, at least one C₄-alkylaminoethylmethacrylate, and at least one C₈ alkyl acrylamide.

The additional film-forming and/or setting polymers can preferably be present in the cosmetic agents according to the invention in an amount of 0.1 wt. % to 12.0 wt. %, especially of 0.2 wt. % to 10.0 wt. %, and in particular of 0.5 wt. % to 8.0 wt. %, in each case based on the total weight of the cosmetic agent.

So as to intensify the effect according to the invention, the cosmetic agents according to the invention preferably additionally comprise at least one surfactant. Surfactants within the meaning of the present invention are amphiphilic (bifunctional) compounds, which are composed of at least one hydrophobic molecule part and at least one hydrophilic molecule part. The hydrophobic group is preferably a hydrocarbon chain having 8 to 28 carbon atoms, which can be saturated or unsaturated, linear or branched. This C₈ to C₂₈ alkyl chain is particularly preferably linear. The hydrophilic molecule part is preferably a polyethylene oxide group having at least 2 EO units or an acid group. A basic property of the surfactants and emulsifiers is the oriented absorption at interfaces, the aggregation into micelles, and the formation of lyotropic phases.

Within the scope of the present invention, the cosmetic agents can additionally comprise at least one surfactant from the group of non-ionic surfactants, anionic surfactants, amphoteric surfactants, and the mixtures thereof, preferably non-ionic surfactants. The group of ampholytic or also amphoteric surfactants comprises zwitterionic surfactants and ampholytes.

It has been found to be particularly preferred for the agents according to the invention to additionally include at least one non-ionic surfactant. Non-ionic surfactants include a polyol group, a polyalkylene glycol ether group or a combination of a polyol and polyglycol ether group, for example, as the hydrophilic group.

Addition products of alkylene oxide to saturated linear fatty alcohols and fatty acids, each having 2 to 100 moles ethylene oxide per mole of fatty alcohol or fatty acid, have proven to be particularly preferred non-ionic surfactants. Cosmetic agents according to the invention having excellent properties are obtained if these comprise, as non-ionic surfactants, C₁₂-C₃₀ fatty acid monoesters and diesters of addition products of 1 to 30 moles ethylene oxide to glycerol and/or addition products of 5 to 60 moles ethylene oxide to castor oil and hydrogenated castor oil in an amount of 0.1 to 5 wt. %, especially of 0.2 to 3 wt. %, and in particular of 0.2 to 1 wt. %, based on the total weight of the cosmetic agent.

In principle, all anionic surface-active substances that are suitable for use on the human body are suitable anionic surfactants. These are characterized by a water-soluble-rendering anionic group, such as a carboxylate, sulfate, sulfonate or phosphate group and a lipophilic alkyl group having approximately 8 to 30 carbon atoms. In addition, glycol or polyglycol ether groups, ester, ether and amide groups and hydroxyl groups can be present in the molecule. Examples of suitable anionic surfactants, each in the form of the sodium, potassium and ammonium salts, and monoalkanol, dialkanol and trialkanol ammonium salts having 2 to 4 carbon atoms in the alkanol group, are:
linear and branched fatty acids having 8 to 30 carbon atoms (soaps);
ether carboxylic acids of formula R²²—O—(CH₂—CH₂O)ₓ—CH₂—COOH, in which R²² is a linear alkyl group having 8 to 30 carbon atoms, and x=0 or 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid monoalkyl and dialkyl esters having 8 to 24 carbon atoms in the alkyl group, and sulfosuccinic acid monoalkyl polyoxyethyl esters having 8 to 24 carbon atoms in the alkyl group and 1 to 6 oxyethyl groups;
linear alkane sulfonates having 8 to 24 carbon atoms;
linear alpha-olefin sulfonates having 8 to 24 carbon atoms;
alpha-sulfo fatty acid methyl esters of fatty acids having 8 to 30 carbon atoms;
alkyl sulfates and alkyl polyglycol ether sulfates of formula R²³—O(CH₂—CH₂O)ₓ—OSO₃H, in which R²³ is a preferably linear alkyl group having 8 to 30 carbon atoms, and x=0 or 1 to 12;
sulfated hydroxyalkyl polyethylene and/or hydroxyalkylenepropylene glycol ethers;
sulfonates of unsaturated fatty acids having 8 to 24 carbon atoms and 1 to 6 double bonds;
sulfated fatty acid alkylene glycol esters of formula R²⁴CO(AlkO)ₙSO₃M, in which R²⁴CO— denotes a linear or branched, aliphatic, saturated and/or unsaturated acyl group having 6 to 22 carbon atoms, Alk denotes CH₂CH₂, CHCH₃CH₂ and/or CH₂CHCH₃, n denotes numbers from 0.5 to 5, and M denotes a cation, as they are described in the unexamined patent application DE 19736906;
amide ether carboxylic acids.

Cosmetic agents of the present embodiment that are preferred according to the invention include the additional at least one surfactant in a total amount of 0.01 wt. % to 5 wt. %, and in particular of 0.05 wt. % to 0.5 wt. %, based on the total weight of the cosmetic agent.

Within the scope of the present invention, it is preferred for the cosmetic agents according to the invention to be formulated as aerosol sprays or non-aerosol sprays. An “aerosol spray” shall be understood to mean a cosmetic product in which the cosmetic agent according to the invention is present in pressurized form in a pressure-resistant vessel and sprayed by way of propellant gas. In contrast, cosmetic products in the form of a “non-aerosol spray” are present in a vessel that is under normal pressure and are sprayed as an atomized spray by way of mechanical action using a pump or squeeze system.

If the cosmetic agent according to the invention is present in the form of an aerosol spray, this preferably comprises at least one propellant in a total amount of 10 to 80 wt. %, especially 20 to 70 wt. %, and in particular 30 to 60 wt. %, based on the total weight of the cosmetic agent. The sizes of the aerosol droplets and the respective size distribution can be set for a particular spraying device by way of the quantity ratio of propellant to the remaining components of the cosmetic agents. The spray rate of the spray according to the invention is preferably 6.5 to 10.0 g/10 s.

Suitable propellants according to the invention are selected, for example, from N₂O, dimethylether, CO₂, air, alkanes having 3 to 5 carbon atoms, such as propane, n-butane, iso-butane, n-pentane and iso-pentane, and the mixtures thereof. Dimethylether, propane, n-butane, iso-butane and mixtures thereof are preferred. According to a preferred embodiment, the aforementioned alkanes, mixtures of the aforementioned alkanes, or mixtures of the aforementioned alkanes with dimethylether are used as the only propellant. The present invention, however, explicitly also covers the simultaneous use of propellants of the chlorofluorocarbon type, and in particular of the fluorocarbons.

The propellant (in particular dimethylether) is preferably present in the agents according to the invention in the embodiment as an aerosol spray in a total amount of 30 to 60 wt. %, based on the total weight of the cosmetic agent.

Dimethylether or mixtures of propane and butane are especially particularly preferably used as the only propellant in a weight ratio of propane to butane of 20:80 to 15:85. The mixtures, in turn, are preferably used in the agents according to the invention in a total amount of 30 to 55 wt. %, based on the total weight of the cosmetic agent. According to the invention, butane shall be understood to cover n-butane, iso-butane and mixtures of n-butane and iso-butane. Most preferably, dimethylether is used as the only propellant.

If the cosmetic agents according to the invention are present in the form of a “non-aerosol spray,” these agents can be delivered using any arbitrary propellant-free spray system that comprises a dispensing vessel and a spray valve, which is to say, for example, in a flexible pressurized bottle comprising a dip tube and a spray valve (squeeze bottle), in a balloon-shaped atomizer operating according to the Venturi principle, or in a pump spray bottle, having a pump lever that is operated by way of the index finger or the entire hand in the manner of a trigger. In an embodiment that is preferred for cosmetic use, the dispensing vessel comprises a manually operated spray pump.

According to CAS nomenclature, the structural units of formulas (Ia), (Ha), (IIIa), (IVa), (Va) are described as adipic acid, 1,6-hexanediol, nepentylglycol, isophorone dicyanate, isophorone diamine, and N-(2-aminoethyl)-3-aminoethane sodium sulfonate. Furthermore according to CAS nomenclature, the structural units of formulas (VIa), (VIIa) and (VIIIa) are described as polyethylene-polypropylene glycol-bis(2-methyl-2-propene-1-yl)ether, 3-[(2-aminoethyl)amino]propyl-siloxane and dimethylsiloxane. A further subject matter of the present invention is thus a cosmetic agent for temporarily reshaping keratin fibers, comprising, in a cosmetically compatible carrier,
a) at least one anionic polyurethane polymer of adipic acid, 1,6-hexanediol, nepentylglycol, isophorone dicyanate, isophorone diamine and the sodium salt of N-(2-aminoethyl)-3-aminoethane sulfonic acid; and
b) at least one silicone-containing cationic copolymer of polyethylene-polypropylene glycol-bis(2-methyl-2-propene-1-yl)ether, 3-[(2-aminoethyl)amino]propyl-siloxane and dimethylsiloxane.

Surprisingly, the above-mentioned combination of the specific anionic polyurethane polymer and of the specific silicone-containing cationic copolymer does not result in a negative interaction, so that the cosmetic agents according to the invention exhibit both the outstanding straightening properties of the anionic polyurethane polymer and the outstanding protective properties against hair damage of the cationic silicone-containing copolymer. The excellent compatibility of these two specific polymers moreover results in extremely storage-stable cosmetic agents according to the invention.

What was said above with respect to the cosmetic agents according to the invention applies, mutatis mutandis, to further preferred embodiments.

Moreover, a further subject matter of the present invention is a method for temporarily reshaping keratin fibers, wherein the method comprises the following method steps:
a) providing a cosmetic agent according to the invention;
b) applying the cosmetic agent according to the invention to the keratin fibers;
c) distributing the applied cosmetic agent according to the invention on the keratin fibers; and
d) reshaping the keratin fibers at a temperature of 30° C. to 250° C. to the desired shape.

Within the scope of the method according to the invention, it may be provided that the reshaping of the keratin fibers is carried out at a temperature of 40° C. to 250° C., especially of 50 to 250° C., preferably of 80° C. to 250° C., and in particular of 120° C. to 250° C. It is preferred according to the invention if the reshaping is carried out using a hot air blow dryer or a straightening iron, in particular a straightening iron. In this way, the keratin fibers can be heated during the reshaping process. However, it may also be provided that the keratin fibers are heated after the cosmetic agent according to the invention has been applied and/or has been distributed.

The method according to the invention yields an outstanding straightening result, however without resulting in damage to the hair due to the high temperatures necessary during straightening of the keratin fibers. The hair straightened by way of the method according to the invention has an outstanding and long-lasting hold of the hair style and outstanding wet and/or dry combability and care.

What was said above with respect to the cosmetic agents according to the invention applies, mutatis mutandis, with respect to further preferred further embodiments of the method according to the invention.

Finally, a further subject matter of the present invention is the use of a cosmetic agent according to the invention for temporarily reshaping keratin fibers at a temperature of 30° C. to 250° C.

According to a preferred embodiment of this subject matter, a temperature of 40° C. to 250° C., especially of 50 to 250° C., preferably of 80° C. to 250° C., and in particular of 120° C. to 250° C. is used for temporarily reshaping the keratin fibers. What was said with respect to the cosmetic agents according to the invention and with respect to the method according to the invention applies, mutatis mutandis, with respect to further preferred embodiments of the use according to the invention.

The following examples describe the present invention in more detail, without limiting it to these examples.

### Examples

1. Formulations (all information is in percent by weight, based on the total weight of the particular cosmetic agent):

E4

| Raw material | E1 | E2 | E3 | E4 |
|---|---|---|---|---|
| Anionic polyurethane polymer¹⁾ | 0.5* | 1.0* | 1.2* | 1.5* |
| Cationic silicone-containing | 0.2* | 0.2* | 0.5* | 0.5* |
| copolymer²⁾ | | | | |
| Anionic copolymer³⁾ | 0.2* | — | 0.1* | — |
| Ethanol | 5.0 | 5.0 | 5.0 | 5.0 |
| Propylene glycol | 1.0 | 1.0 | 1.0 | 1.0 |
| PEG-40 Hydrogenated Castor Oil | 0.5 | 0.5 | 0.5 | 0.5 |
| Perfume | 0.15 | 0.15 | 0.15 | 0.15 |
| Lactic acid | to make | to make | to make | to make |
| | pH 4.5 | pH 4.5 | pH 4.5 | pH 4.5 |
| Water | to make | to make | to make | to make |
| | 100 | 100 | 100 | 100 |

¹⁾including structural units of formulas (la) to (Va) where a = 4, b = 6 and X⁺ = Na; INCI name: Polyurethane-48
²⁾including structural units of formulas (Vla) to (VIIIa) where c = 20 and d = 35; INCI name: Bis-Isobutyl PEG/PPG-20/35/Amodimethicone Copolymer
³⁾INCI name: Sodium Laneth-40 Maleate/Styrene Sulfonate Copolymer
*active substance content

The cosmetic agents E1 to E4 were obtained by mixing the above ingredients.

Thereafter, the cosmetic agents were applied to keratin fibers, and these fibers were straightened using a straightening iron at temperatures between 150° C. and 240° C. All cosmetic agents resulted in an outstanding straightening action and effective protection against damage to the keratin fibers due to the high temperature and in good care, in particular wet and/or dry combability of the keratin fibers.

Damage to the hair after straightening, using a cosmetic agent E5 according to the invention and a comparison composition V1, was determined as follows by ascertaining the hair breakage after straightening (information in the table below is in wt. %, based on the total weight of the cosmetic agent):

| Raw material | E5 | V1 |
|---|---|---|
| Anionic polyurethane polymer¹⁾ | 0.5* | — |
| Cationic silicone-containing | 0.2* | — |
| copolymer²⁾ | | |
| Ethanol | 5.0 | 5.0 |
| Propylene glycol | 0.5 | 0.5 |
| PEG-40 Hydrogenated Castor Oil | 0.3 | 0.3 |
| Perfume | 0.2 | 0.2 |
| Lactic acid | to make pH 4.5 | — |
| Phosphoric acid 85% | — | to make pH 4.5 |
| Water | to make 100 | to make 100 |

¹⁾including structural units of formulas (la) to (Va) where a = 4, b = 6 and X⁺ = Na; INCI name: Polyurethane-48
²⁾including structural units of formulas (Vla) to (VIIIa) where c = 20 and d = 35; INCI name: Bis-Isobutyl PEG/PPG-20/35/Amodimethicone Copolymer

10 strands of hair we prepared for each composition. Initially, the strands of hair (IHIP—New York, European natural hair 6/0, lot #03/2013, p. 78, length: 12 cm, weight: 1±0.05 g) were cleaned in a 3 wt. % solution of Texapon NSO in water for 5 minutes in an ultrasonic bath and washed out with tap water for 5 minutes while combing. After the strands were dried using a towel, 0.5 g of the respective composition per gram of strand of hair was applied and massaged in. Afterwards, the strands of hair were automatically combed (comb: Hercules Sagemann, fine-tooth hard rubber comb) and at the same time dried using a blow dryer at 110° C. for 5 minutes. All strands were stored at 25° C. and 25% relative humidity for at least 24 hours and then equilibrated to 25° C. and 50% relative humidity for at least 1 hour and weighed.

Each strand was combed 20,000 times under constant ambient conditions (25° C., 50% relative humidity), and the broken hair was collected in a box underneath the comb. The amount of broken hair was weighed and related to the weight of the particular strand of hair prior to combing. All results were reviewed by way of statistical tests (Shapiro-Wilk test, Bartlett test, Newman-Keuls test) and were significant. The following results were obtained:

| Composition | Hair breakage [%] |
|---|---|
| V1 | 8.7 |
| E1 | 4.6 |

The use of the combination according to the invention of an anionic polyurethane polymer and a silicone-containing cationic copolymer results in significantly reduced hair breakage, and thus in significantly reduced damage to the hair, during straightening of the hair.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent for temporarily reshaping keratin fibers, comprising: in a cosmetically compatible carrier,
a) at least one anionic polyurethane polymer, comprising at least one structural unit of formula (I), at least one structural unit of formula (II), at least one structural unit of formula (III), at least one structural unit of formula (IV), and at least one structural unit of formula (V) 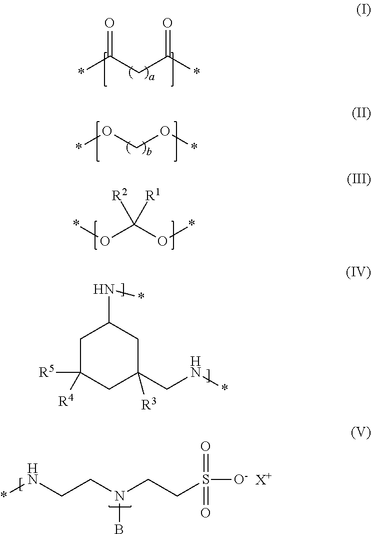
where
a and b, independently of one another, denote integers from 1 to 20;
R¹, R², R³, R⁴ and R⁵, independently of one another, denote a hydrogen atom or a C₁ to C₄ alkyl group;
B denotes a hydrogen atom or a direct bond with a further structural unit;
X⁺ denotes a physiologically compatible cation; and
b) at least one silicone-containing cationic copolymer, comprising at least one structural unit of formula (IV), at least one structural unit of formula (VII), and at least one structural unit of formula (VIII) 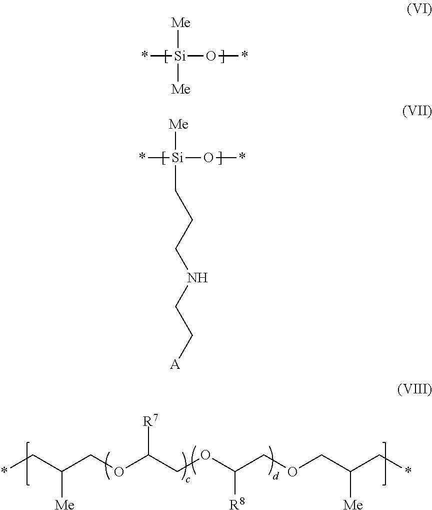
where
A denotes a hydroxyl group, an amino group or a thiol group;
R⁷ and R⁸, independently of one another, denotes a hydrogen atom, a methyl group, an ethyl group, a C₁-C₄ aralkyl group, C₂-C₆ alkenyl group, or a C₂-C₆ hydroxyalkyl group; and
c and d, independently of one another, denote integers from 10 to 55.

2. The cosmetic agent according to claim 1, wherein the structural unit of formula (I) a denotes integers from 1 to 10, and in the structural unit of formula (II) b denotes integers from 1 to 12.

3. The cosmetic agent according to claim 1, wherein the structural unit of formula (I) a denotes integers 2 to 6, and in the structural unit of formula (II) b denotes integers 4 to 8.

4. The cosmetic agent according to claim 1, wherein the at least one anionic polyurethane polymer a) comprises least one structural unit of formula (Ia), at least one structural unit of formula (IIa), at least one structural unit of formula (IIIa), at least one structural unit of formula (IVa) and at least one structural unit of formula (Va) 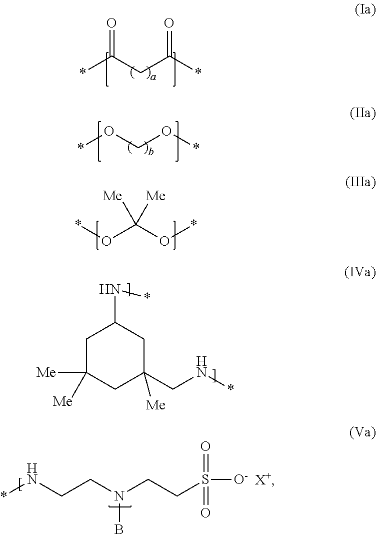
where
a denotes integers from 2 to 6;
b denotes integers from 4 to 8; and
X⁺ denotes a physiologically compatible cation.

5. The cosmetic agent according to claim 1, wherein the at least one anionic polyurethane polymer a) is present in a total amount of 0.05 to 5 wt. % based on the total weight of the cosmetic agent.

6. The cosmetic agent according to claim 1, wherein the at least one anionic polyurethane polymer a) is present in a total amount of 0.2 to 1.5 wt. %, based on the total weight of the cosmetic agent.

7. The cosmetic agent according to claim 1, wherein the group A in the structural unit of formula (VII) denotes an amino group.

8. The cosmetic agent according to claim 1, wherein the groups R⁷ and R⁸ in the structural unit of formula (VIII), independently of one another, denote a hydrogen atom or a methyl group.

9. The cosmetic agent according to claim 1, wherein the groups R⁷ and R⁸ denotes a methyl group.

10. The cosmetic agent according to claim 1, wherein the structural unit of formula (VIII) c denotes integers from 15 to 30

11. The cosmetic agent according to claim 1, wherein the structural unit of formula (VIII) c denotes 25 to 40.

12. The cosmetic agent according to claim 1, wherein the at least one silicone-containing cationic copolymer b) comprises at least one structural unit of formula (VIa), at least one structural unit of formula (VIIa) and at least one structural unit of formula (VIIIa) 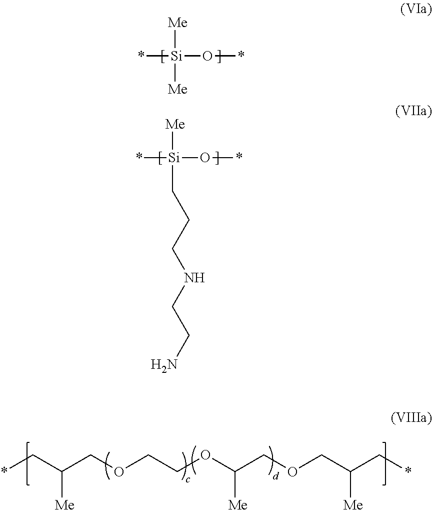
where
c denotes integers from 15 to 25; and
d denotes integers from 25 to 40.

13. The cosmetic agent according to claim 1, wherein the at least one silicone-containing copolymer b) is present in a total amount of 0.01 to 2 wt. % based on the total weight of the cosmetic agent.

14. The cosmetic agent according to claim 1, wherein the at least one silicone-containing copolymer b) is present in a total amount of 0.05 to 0.5 wt. %, based on the total weight of the cosmetic agent.
a) The cosmetic agent according to claim 1, wherein the at least one anionic polyurethane polymer comprises at least one structural unit of formula (Ia), at least one structural unit of formula (IIa), at least one structural unit of formula (IIIa), at least one structural unit of formula (IVa), and at least one structural unit of formula (Va) 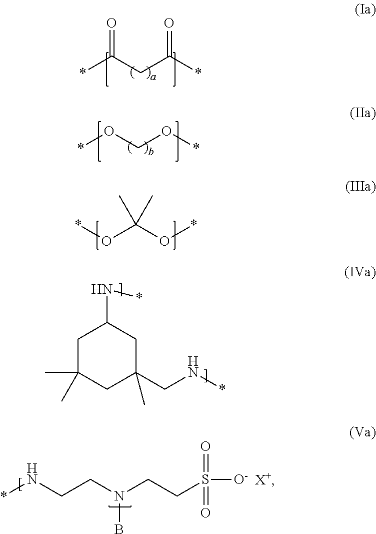
where
a denotes integers from 2 to 6;
b denotes integers from 4 to 8; and
X⁺ denotes metal cations of the physiologically compatible metals from groups IA, IB, IIA, IIB, IIIB, VIA or VIII of the periodic system of elements, ammonium ions, and cationic organic compounds comprising a quaternized nitrogen atom; and
b) at least one cationic silicone-containing copolymer that comprises at least one structural unit of formula (VIa), at least one structural unit of formula (VIIa) and at least one structural unit of formula (VIIIa) 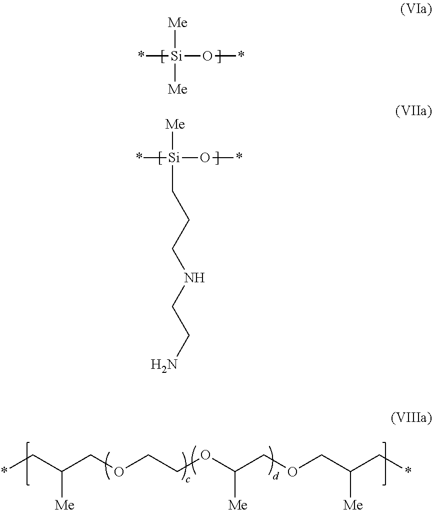
where
c denotes integers from 15 to 25; and
d denotes integers from 25 to 40.

15. The cosmetic agent according to claim 1, wherein the cosmetic agent has a weight ratio of the total amount of the at least one anionic polyurethane polymer a) to the total amount of the at least one silicone-containing copolymer b) of 300:1 to 1:40.

16. The cosmetic agent according to claim 1, wherein the cosmetic agent has a weight ratio of the total amount of the at least one anionic polyurethane polymer a) to the total amount of the at least one silicone-containing copolymer b) of 10:1 to 2:1.

17. The cosmetic agent according claim 1, wherein the cosmetic agent additionally comprises an anionic polymer, comprising at least one structural unit of formula (IX) and at least one structural unit of formula (X) 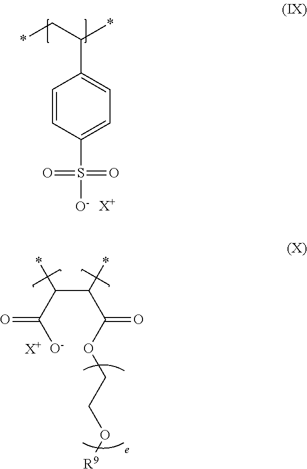
where
R⁹ denotes a linear or branched, saturated or unsaturated C₁₀-C₄₀ alkyl group or a C₁₀-C₄₀ hydroxyalkyl group;
e denotes integers between 20 and 70; and
X⁺ denotes a physiologically compatible cation.

18. A cosmetic agent for temporarily reshaping keratin fibers, comprising in a cosmetically compatible carrier
a) at least one anionic polyurethane polymer of adipic acid, 1,6-hexanediol, nepentylglycol, isophorone dicyanate, isophorone diamine and the sodium salt of N-(2-aminoethyl)-3-aminoethane sulfonic acid; and
b) at least one silicone-containing cationic copolymer of polyethylene-polypropylene glycol-bis(2-methyl-2-propene-1-yl)ether, 3-[(2-aminoethyl)amino]propyl-siloxane and dimethylsiloxane.

19. A method for temporarily reshaping keratin fibers, the method comprising the following method steps:
a) providing a cosmetic agent according to claim 1;
b) applying the cosmetic agent to keratin fibers;
c) distributing the applied cosmetic agent on the keratin fibers; and
d) reshaping the keratin fibers at a temperature of 30° C. to 250° C. to the desired shape.
